(19) United States
(12) Patent Application Publication
(10) Pub. No.: US 2022/0322707 A1
(43) Pub. Date: Oct. 13, 2022

(54) Drinkable Supplement Composition for Improved Health and Hydration

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/634,671

(22) PCT Filed: Aug. 14, 2020
(86) PCT No.: PCT/US2020/046409

§ 371 Date: Feb. 11, 2022

Related U.S. Application Data
(60) Provisional application No. 62/886,460, filed on Aug. 14, 2019.

(51) Int. Cl. A23L 2/52; A61K 35/742; A61K 35/747; A61K 47/02; A61K 45/06

(52) U.S. Cl. CPC A23L 2/52; A61K 35/742; A61K 35/747; A61K 47/02; A61K 45/06; A61K 2035/115

(57) ABSTRACT

The present invention provides compositions and methods for enhancing the health of a subject by, for example, restoring the subjects gut microbiome, increasing water and electrolyte absorption in the digestive tract, and/or providing a therapeutic substance to the subject. In certain specific embodiments, the present invention can be used for ameliorating the adverse physiological effects that can result from the presence of one or more detrimental microorganisms in a subjects gut. In certain specific embodiments, the present invention can also be used as an enhanced mode of hydration or re-hydration, particularly for subjects suffering from, for example, a condition that causes vomiting and/or diarrhea.

### CROSS-REFERENCE TO A RELATED APPLICATION

This application claims the benefit of U.S. provisional application Ser. No. 62/886,460, filed Aug. 14, 2019, which is incorporated herein by reference in its entirety.

### BACKGROUND OF THE INVENTION

The gut microbiota is an essential ecosystem within the human body that participates in a large number of vital functions. This system contains a variety of taxa, including bacteria, eukaryotes, viruses, and archaea. Trillions of these organisms build a complex symbiotic relationship, potentially providing a number of benefits to the host, including aiding in normal physiological functions and disease resistance (Clemente 2012).

The composition of each person's microbiota is unique. Twins have been shown to share less than 50% similarity of bacterial taxa, and even less viral similarity (Turnbaugh 2010). Many of the species present in the gut microbiome are bacteria belonging predominately to the phylotypes Bacteroidetes and Firmicutes, while some others, including Actinobacteria, Proteobacteria and Verrucomicrobia, are minor constituents. Methanogenic archaea (mainly Methanobrevibacter smithii), eukaryotes (mainly yeasts) and viruses (mainly phages) can also be present. While these groups tend to be universally present within the gut microbiome, their relative proportions and the particular species vary between individuals (Luzopone 2012). This difference may be determined by genetic influences (Benson 2010).

An individual's health is closely linked to the health of the gut microbiome. In other words, changes in the gut microbiome are often either a cause or an effect of changes in an individual's health. For example, intestinal microflora play an important role in metabolic functions, as well as digestive, immune, endocrine and cardiovascular system functions. When the gut microbiome is disrupted or unbalanced, for example, due to illness, dysbiosis, the presence and/or overgrowth of pathogenic and/or commensal organisms, the presence of a parasite, the use of antibiotics, a food allergy or sensitivity, the implementation of a colonoscopy, or some other influence, these and other body system functions can also be disrupted.

Some symptoms and/or conditions associated with disrupted gut microbiomes, such as vomiting and/or diarrhea, can also lead to dehydration. If not remedied, dehydration can cause loss of appetite, low energy, lack of mental acuity, disorientation and dizziness, and a range of other physiological symptoms, including disruption of the gut microbiome. Dehydration can also be caused by strenuous exercise, exposure to sunlight and heat, and lack of fluid intake.

Currently, the most common way to repair microbiota is usage of probiotics, such as L. acidophilus, L. casei, L. rhamnous, L. bulgaricus, B. bifidum, S. thermophilus, B. coagulans, and others. Usually, probiotics contain one or several live strains of bacteria that are capable of temporarily populating the gut and providing relief to symptoms such as diarrhea, constipation, bloating or nausea.

An additional method of repairing the gut microbiome is through fecal bacteriotherapy, or fecal transplantation. This method involves introducing saline-diluted fecal matter from a healthy donor into a patient's gastrointestinal tract via a nasoduodenal cathether or enema. In many cases, the donor is a relative of the patient. Thus far, fecal transplant has primarily been used to treat Clostridium difficile enterocolitis; however, this method has not been as effective for treating other diseases, such as IBS and Crohn's disease. Additionally, the method is limited by the ability to find a healthy donor, the safety of the method, side effects including abdominal discomfort, bloating, flatulence, diarrhea, constipation, borborygmia, vomiting, transient fever, bleeding and bacteremia, and risk of transmission of infection and/or some chronic diseases.

When the microbiome of an individual is disrupted or unbalanced, or when an individual is afflicted with a disease or condition that causes such disruption or imbalance, the individual would benefit from improved methods of rehydration and restoration of the gut microbiome to a balanced state.

### BRIEF SUMMARY OF THE INVENTION

The present invention provides compositions and methods for enhancing the health of a subject in one or more ways, such as, for example, by restoring the subject's gut microbiome, improving cardiovascular health, and/or increasing water, nutrient, and electrolyte absorption in the digestive tract.

In preferred embodiments, an ingestible composition is provided, comprising water and one or more biological amphiphilic molecules, wherein the amount of the one or more biological amphiphilic molecules is an amount sufficient to lower the surface tension of the water to about 45+/−10 mN/m.

In one embodiment, the one or more biosurfactants are present in the composition in critical micelle concentration (CMC). In certain embodiments, the concentration of biosurfactants in the composition is about 5 ppm to about 30 ppm.

In one embodiment, the biological amphiphilic molecules are microbial biosurfactants. In a specific embodiment, the microbial biosurfactants are selected from, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins and glycerophospholipids), fatty acid ester compounds, and high molecular weight polymers such as lipoproteins, lipopolysaccharide-protein complexes, and polysaccharide-protein-fatty acid complexes.

Biosurfactants used according to the subject invention can have antimicrobial, anti-biofilm, antiviral, anti-inflammatory and immune-modulating properties. Biosurfactants can also increase the solubility and bioavailability of water, nutrients and other health-promoting compounds in the digestive system to promote increased absorption and transport into, for example, the circulatory system.

In some embodiments, the biological amphiphilic molecules are in crude form, meaning they are present in a supernatant resulting from cultivation of a biosurfactant-producing microorganism. The crude form can optionally comprise residual nutrients, other microbial growth by-products, and even microorganisms and/or cellular components.

In some embodiments, the biological amphiphilic molecules are extracted from the products of cultivation and isolated and/or purified.

The one or more biological amphiphilic molecules can further include any one or a combination of: a modified form, derivative, fraction, analog, isoform, isomer or subtype of a biosurfactant, including forms that are biologically or synthetically modified.

In certain embodiments, the composition comprises one or more electrolytes, for example, sources of sodium, potassium, phosphate, bicarbonate, sulfate, chloride, calcium and/or magnesium. In certain preferred embodiments, the one or more electrolytes, when present, are included at a concentration of about 1 mEq/l to about 50 mEq/L.

In certain embodiments, the ingestible composition can further comprise one or more beneficial microorganisms and/or growth by-products thereof. The microorganisms can be viable or in an inactive form. They can be in the form of vegetative cells, spores, conidia, mycelia and/or a combination thereof.

In some embodiments, the microorganisms are added in the form of a culture comprising residual growth medium and/or nutrients from cultivating the microorganism, and/or any growth by-products produced by the microorganism. These can include, for example, enzymes, biopolymers, solvents, acids, proteins, amino acids, polyketides, biosurfactants, or other metabolites that can be useful for, for example, hydration, gut health, cardiovascular health, control of undesirable microorganisms and/or growth of beneficial microorganisms.

Preferably, the beneficial microorganisms are in the form of live cells and/or spores that are capable of germinating and/or surviving in the digestive tract, and furthermore, that promote and/or improve the health of the subject and/or the subject's gut microbiome. For example, in one embodiment, the beneficial microorganisms can be Bacillus spp., Bacteroidetes spp., Bifidobacterium spp. and/or Lactobacillus spp. bacteria. The microbes can be present at a concentration of, for example, at least 1×10⁶ to 1×10¹³ CFU/ml.

In a specific embodiment, the microorganism is Bacillus subtilis natto, Bacillus amyloliquefaciens (e.g., B. amyloliquefaciens NRRL B-67928), or Bacillus coagulans.

In certain embodiments, the ingestible composition further comprises nattokinase, an enzyme produced by Bacillus subtilis natto. Nattokinase is a dietary supplement that can, in some embodiments, dissolve atherosclerotic plaques and support blood circulation, among other health benefits.

The present composition is preferably formulated as a drinkable fluid. Accordingly, in certain embodiments, the composition can further comprise health-promoting components that are, for example, sources of energy, nutrients, vitamins, minerals, herbal extracts, proteins, amino acids, antioxidants, enzymes and/or other health-promoting supplements; as well as additives such as, for example, flavorings, preservatives, prebiotics, pH adjusters, sweeteners and/or dyes. The composition may also be carbonated.

In one embodiment, the composition is formulated as a dehydrated powder or concentrate that can be reconstituted into a drinkable fluid by the addition of water.

In preferred embodiments, the present invention further provides methods of enhancing the health of a subject in need thereof by, for example, restoring the subject's gut microbiome, increasing water and electrolyte absorption in the digestive tract, and/or providing a therapeutic substance to the subject to treat and/or prevent a disease, disorder or health condition.

In certain embodiments, the subject's gut microbiome has been disrupted or imbalanced, due to, for example, infection by a pathogenic microorganism, illness, aging, dietary factors (e.g., food sensitivities, changes in eating and/or nutritional habits), immune system changes, treatment with antibiotics, radiation treatment or exposure, or procedures, such as appendectomies and/or colonoscopies.

In certain embodiments, the subject is dehydrated due to, for example, a disrupted gut microbiome, physical exertion, heat exhaustion, a condition that causes vomiting or diarrhea, and/or as a side-effect of a medical treatment, such as a drug or radiation therapy.

In certain embodiments, the subject has, or is at risk of having, a disease, disorder or health condition affecting, e.g., the cardiovascular system or the endocrine system, for example, diabetes (1 or 2) and/or cardiovascular disease.

In certain embodiments, the methods comprise administering a composition of the present invention to the subject, preferably by ingestion. The optimal rate of administration can depend upon, for example, the physiological characteristics of the subject, the nature or extent of dehydration and/or gut microbiome dysbiosis, and the existence and/or extensiveness of other health conditions. An exemplary rate of application would be approximately 8 to 24 fluid ounces every 4 hours to every 24 hours.

In some embodiments, the methods result in restoration of the subject's gut microbiome, e.g., balancing an unbalanced gut microbiome, regardless of whether the imbalance is a cause or an effect of a disease or another change to the subject's health status. Restoration preferably comprises decreasing the number of overgrown commensal microorganisms, pathogenic microorganisms, and/or biofilms in the GI tract, and/or increasing the number of beneficial microorganisms in the GI tract.

In certain embodiments, the methods result in reduction of one or more undesirable microorganisms due to the action of the one or more beneficial microorganisms and/or microbial growth by-products of the ingestible composition, which outcompete and/or directly control the undesirable microorganism(s) and/or biofilms.

In some embodiments, the methods result in improved hydration and/or nutrient absorption for a subject who is dehydrated and/or depleted of a certain nutrient, or is at risk of becoming dehydrated and/or nutrient-depleted, due to, for example, prolonged physical activity, illness, or cancer treatments. In certain embodiments, the methods result in replenishment of electrolytes in the subject.

In certain embodiments, the present invention can be used to enhance a subject's overall health and well-being. In one embodiment, the present invention can be used to enhance the functioning of a body system, tissue or organ, such as metabolic functions, the digestive system, the immune system, the endocrine system, and/or the cardiovascular system.

In a specific embodiment, the methods result in improved cardiovascular health due to, for example, reduction in artherosclerotic plaques and improved blood circulation.

In one embodiment, the present invention can be used to treat and/or prevent a disease, disorder or health condition that is a cause and/or a result of a disrupted or unbalanced gut microbiome, such as, for example, dehydration, Irritable Bowel Syndrome, Type 1 diabetes, other autoimmune disorders, colorectal cancer, and neurodevelopmental and neurodegenerative diseases.

### DETAILED DESCRIPTION

The present invention provides compositions and methods for enhancing the health of a subject in one or more ways, such as, for example, by restoring the subject's gut microbiome, improving cardiovascular health, and/or increasing water, nutrient, and electrolyte absorption in the digestive tract.

### Selected Definitions

As used herein, an “isolated” or “purified” nucleic acid molecule, polynucleotide, polypeptide, protein, organic compound such as a small molecule, or other compound is substantially free of other compounds, such as cellular material, genes or gene sequences, and amino acids or amino acid sequences, which may flank it in its naturally-occurring state. A purified or isolated microbial strain is removed from the environment in which it exists in nature and/or in which it was cultivated. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, acids, solvents, alcohols, proteins, carbohydrates, vitamins, minerals, microelements, amino acids, polymers, and surfactants.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be absent, or present at, for example, a concentration of at least 1×10⁴, 1×10⁵, 1×10⁶, 1×10², 1×10⁸, 1×10⁹, 1×10¹⁰, 1×10¹¹, 1×10¹², 1×10¹³ or more CFU per milliliter of the composition.

The present invention further provides “microbe-based products,” which are products that are to be applied in practice to achieve a desired result. The microbe-based product can be simply a microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, the term “plurality” refers to any number or amount greater than one.

As used herein “preventing” or “prevention” of a disease, condition or disorder means delaying, inhibiting, suppressing, forestalling, and/or minimizing the onset or progression of a particular sign or symptom thereof. Prevention can include, but does not require, indefinite, absolute or complete prevention throughout a subject's lifetime, meaning the sign or symptom may still develop at a later time and/or with a lesser severity than it would without preventative measures. Prevention can include reducing the severity of the onset of such a disease, condition or disorder, and/or inhibiting the progression of the condition or disorder to one that is more severe. Furthermore, prevention can include reducing the risk to a subject of acquiring or developing a particular disease, condition or disorder.

As used herein, the term “reduces” means a negative alteration, and the term “increases” means a positive alteration, wherein the negative or positive alteration is an alteration of at least 1%, 5%, 10%, 15%, 20%, 25%, 30%, 35%, 40%, 45%, 50%, 55%, 60%, 70% 75%, 80%, 85%, 90%, 95%, 99% or 100%.

As used herein, the term “reference” means a standard or control condition.

As used herein, the term “subject” refers to an animal, such as a mammal. The animal may be for example, pigs, horses, goats, cats, mice, rats, dogs, primates, e.g., apes, chimpanzees and orangutans, guinea pigs, hamsters, cows, sheep, birds, e.g., chickens, reptiles, fish, as well as any other vertebrate or invertebrate. The preferred subject in the context of this invention is a human of any gender. The subject can be of any age or stage of development, including infant, toddler, adolescent, teenager, adult, middle-aged and senior. In some embodiments, the subject is a middle-aged or elderly adult, e.g., 50 years of age or older.

As used herein, the term “surfactant” means a surface active compound that lowers the surface tension (or interfacial tension) between two liquids, between a liquid and a gas, or between a liquid and a solid. Surfactants act as, e.g., detergents, wetting agents, emulsifiers, foaming agents, and dispersants. A “biosurfactant” is a surface-active substance produced by a living cell.

As used herein, the term “treatment” refers to eradicating, reducing, ameliorating, or reversing a sign or symptom of a health condition, disease or disorder to any extent, and includes, but does not require, a complete cure of the condition, disease or disorder. Treating can be curing, improving, or partially ameliorating a disorder. “Treatment” can also include improving or enhancing a condition or characteristic, for example, bringing the function of a particular system in the body to a heightened state of health or homeostasis.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Compositions

In preferred embodiments, an ingestible composition is provided for promoting the health of a subject by, for example, restoring the subject's gut microbiome, increasing water and electrolyte absorption in the digestive tract, and/or providing a therapeutic substance to the subject to treat and/or prevent a disease, disorder or health condition.

In certain embodiments, the composition comprises water and one or more biological amphiphilic molecules, wherein the amount of the one or more biological amphiphilic molecules is an amount sufficient to lower the surface tension of the water to about 45+/−10 mN/m.

Typically, drinking water has a surface tension above 70 mN/m, or around 73-75 mN/m. The surface tension of human blood, however, is in the rang of 50 to 55 mN/m. By reducing the surface tension of water in the composition, the biological amphiphilic molecules increase the bioavailability of water molecules and other compounds that may be dissolved and/or dispersed therein.

In one embodiment, the biological amphiphilic molecules are microbial biosurfactants. Biosurfactants are a structurally diverse group of surface-active substances produced by microorganisms. Biosurfactants are biodegradable and can be produced using selected organisms on renewable substrates. Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g., W. anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondii, P. occidentalis); Starmerella spp. (e.g., S. bombicola); and so on.

All biosurfactants are amphiphiles. They consist of two parts: a polar (hydrophilic) moiety and non-polar (hydrophobic) group. The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), by organic acids in the case of flavolipids, or, in the case of glycolipids, by a carbohydrate.

Due to their amphiphilic structure, biosurfactants increase the surface area of hydrophobic water-insoluble substances, increase the water bioavailability of such substances, and can change the properties of bacterial cell surfaces. Biosurfactants accumulate at interfaces, thus reducing interfacial tension and leading to the formation of aggregated micellar structures in solution. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including human health.

In a specific embodiment, the microbial biosurfactants are selected from, for example, glycolipids, lipopeptides, flavolipids, phospholipids, fatty acid ester compounds, and high molecular weight polymers such as lipoproteins, lipopolysaccharide-protein complexes, and polysaccharide-protein-fatty acid complexes.

In one embodiment, the biosurfactants according to the present invention are selected from glycolipids, such as rhamnolipids (RLP), sophorolipids (SLP), cellobiose lipids, trehalose lipids (TL) and/or mannosylerythritol lipids (MEL).

In one embodiment, the biosurfactants are selected from lipopeptides, including, for example, surfactin, iturin, fengycin, arthrofactin, viscosin and/or lichenysin.

In one embodiment, the biosurfactant is a phospholipid selected from, for example, phosphatidic acid, lyso-phosphatidic acid, phosphatidylcholine, lysophosphatidylcholine, phosphatidylglycerol, diphosphatidylglycerol, phosphatidylglycerophosphate, phosphatidylserine, phosphatidylinositol, phosphatidylglycerophosphoglycerol, Bis(monoacylglycero)phosphate (BMP), Bis(diacylglycero)phosphate (BDP), acylphosphatidylglycerol, phosphatidylethanolamine, N-acylphosphatidylethanolamine, and cardiolipins, sphingomyelin, and plasmalogens.

In one embodiment, the one or more biosurfactants are present in the composition in critical micelle concentration (CMC). In certain embodiments, the concentration of biosurfactants in the composition is about 5 ppm to about 30 ppm, or about 10 ppm to about 20 ppm.

In some embodiments, the biological amphiphilic molecules are in crude form, meaning they are present in a supernatant resulting from cultivation of a biosurfactant-producing microorganism. The crude form can optionally comprise residual nutrients, other microbial growth by-products, and even microorganisms and/or cellular components.

In some embodiments, the biological amphiphilic molecules are extracted from the products of cultivation and isolated and/or purified.

The one or more biological amphiphilic molecules can further include any one or a combination of: a modified form, derivative, fraction, analog, isoform, isomer or subtype of a biosurfactant, including forms that are biologically or synthetically modified.

In certain embodiments, the composition comprises one or more electrolytes, for example, sources of sodium, potassium, phosphate, bicarbonate, sulfate, chloride, calcium and/or magnesium. In certain preferred embodiments, the one or more electrolytes, when present, are included at a concentration of about 1 mEq/L to about 50 mEq/L, or about 5 mEg/L to about 35 mEq/L.

In certain embodiments, the composition can comprise one or more beneficial microorganisms. The microorganisms can be viable or in an inactive form. They can be in the form of vegetative cells, spores, conidia, mycelia and/or a combination thereof.

In some embodiments, the microorganisms are added in the form of a culture comprising residual growth medium and/or nutrients from cultivating the microorganism, and/or any growth by-products produced by the microorganism. These can include, for example, enzymes, biopolymers, solvents, acids, proteins, amino acids, polyketides, biosurfactants, or other metabolites that can be useful for, for example, hydration, gut health, cardiovascular health, control of undesirable microorganisms and/or growth of beneficial microorganisms.

Preferably, the beneficial microorganisms are in the form of live cells and/or spores that are capable of germinating and/or surviving in the digestive tract, and furthermore, that promote and/or improve the health of the subject and/or the subject's gut microbiome.

Organisms according to the present invention can include, for example, yeasts, fungi, bacteria and archaea. In preferred embodiments, the microorganisms are bacteria, such as, for example, Bacillus spp., Bacteroides spp., Clostridium spp., Faecalibacterium spp., Eubacterium spp., Ruminococcus spp., Peptococcus spp., Peptostreptococcus spp., Enterococcus spp., Bifidobacterium spp., Lactobacillus spp., Enterobacter spp., Klebsiella spp., and Escherichia spp. The microbes can be present at a concentration of, for example, at least 1×10⁶ to 1×10¹³ CFU/ml.

In a specific embodiment, the microorganism is Bacillus subtilis natto, Bacillus amyloliquefaciens (e.g., B. amyloliquefaciens NRRL B-67928), or Bacillus coagulans.

In certain embodiments, the ingestible composition further comprises nattokinase, an enzyme produced by Bacillus subtilis natto. Nattokinase is a dietary supplement that may dissolve atherosclerotic plaques, support blood circulation, lower blood pressure, and/or provide fibrinolytic therapy to a subject, among other health benefits. The nattokinase can be included at 5 to 300 m mg/dosage, 10 to 200 mg/dosage, or 20 to 100 mg/dosage of the subject composition.

In some embodiments, the composition further comprises prebiotics to support growth of beneficial microbes in the gut. Prebiotics can include, for example, fermentable fibers derived from fructans and xylans, inulin, fructooligosaccharides, xylooligosaccahrides and galactooligosaccharides. Foods known to contain prebiotics include, for example, chicory root, onions, garlic, leek, oatmeal, wheat bran, asparagus, dandelion greens, Jerusalem artichoke, and banana.

In one embodiment, the composition can be formulated as an orally-consumable product and administered orally to an animal or human subject.

Orally-consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally consumable products (particularly food and pharmaceutical products) during their production, treatment or processing and intended to be introduced into the human or animal oral cavity.

Orally-consumable products can also include substances intended to be swallowed by humans or animals and then digested in an unmodified, prepared or processed state; the orally consumable products according to the invention therefore also include casings, coatings or other encapsulations that are intended also to be swallowed together with the product or for which swallowing is to be anticipated.

In preferred embodiments, the present composition is preferably formulated as a drinkable fluid. Accordingly, in certain embodiments, the composition can further comprise components that are, for example, sources of energy, nutrients, vitamins, minerals, herbal extracts, proteins, amino acids, antioxidants, enzymes and/or other health-promoting supplements; as well as additives such as, for example, flavorings, preservatives, prebiotics, pH adjusters, sweeteners and/or dyes. The composition may also be carbonated.

In one embodiment, the composition comprises a vitamin, such as, for example, vitamins A, C, D, E, K, B1 (thiamine), B2 (riboflavin), B3 (niacin), B6, B7 (biotin), B12, folate (or folic acid), panthothenic acid, nicotinic acid, choline chloride, carnitine, inositol and para-amino-benzoic acid. In certain embodiments, the adjuvant composition can help facilitate solubilization of lipophilic vitamins, such as, for example, vitamins A, D, E, and/or K.

In one embodiment, the composition comprises a macro-minerals and/or trace mineral, such as, for example, calcium, phosphorus, magnesium, sodium, potassium, chloride, sulfur, iron, manganese, copper, iodine, zinc, cobalt, fluoride and selenium.

In one embodiment, the composition comprises a supplement, such as, for example, caffeine, Echinacea, fish oil, ginseng, glucosamine, chondroitin sulfate, garlic extract, St. John's Wort, Saw Palmetto, ginko, omega-3 fatty acids, omega-6 fatty acids, melatonin, beta carotene, flavonoids (e.g., anthocyanins), collagen peptides, acai, activated charcoal, alfalfa, arnica, astragalus, aloe vera, ashwagandha, bee pollen, belladonna, berberine, bilberry, betaine, bitter melon, bitter orange, black cohosh, black psyllium, black tea, blessed thistle, blond psyllium, blueberry, blue-green algae, boron, butterbur, calendula, cannabidiol (CBD), capsaicin, capsicum, cartilage, cat's claw, chamomile, chasteberry, chitosan, cinnamon, clove, coconut, cod liver oil, colloidal silver, cranberry, creatine, dandelion, deer velvet, devil's claw, DHEA, Dong Quai, eleuthero, ephedra, eucalyptus, elderberry, evening primrose, fenugreek, feverfew, flaxseed, fucus vesiculosus, ginger, glycyrrhizin, goji, goldenseal, grape, grape seed, grapefruit, green coffee, green tea, guarana, guar gum, gymnema, hawthorn, hemp, hibiscus, honey, honokiol, hoodia, hops, horse chestnut, horny goat weed, horsetail, hydrazine sulfate, kava, kola nut, lavender, lemongrass, licorice root, lutein, lycopene, maca, mangosteen, methylsulfonylmethane, milk thistle, mistletoe, monolaurin, niacinamide, noni, oats, olive, oregano, palm oil, papaya, pau d'arco, peanut oil, pennyroyal, peppermint, pomegranate, propolis, quercetin, rose hip, raspberry ketone, red clover, red yeast rice, reishi mushroom, resveratrol, rose hip, sage, saw palmetto, Satureja bachtiarica oil, senna, slippery elm, soy, spearmint, stevia, tart cherry, tea tree oil, thunder god vine, beetroot, tellimagrandin II, turmeric, valerian, whey protein, wild yam, willow bark, yerba mate, yohimbe, 5-HTP and others.

In one embodiment, the composition comprises an enzyme, such as, for example, coenzyme Q10, lipase, bromelain, papain, chymopapain A, chymopapain B, papaya peptidase A, trypsin, chymotrypsin, proteases, lipases, amylases, pancrelipase, digestive enzymes, lactase, alpha-glactosidase, cellulase, phytase, and beta-glucanase.

Other health-promoting substances may include, but are not limited to, antioxidants, beta-glucans, bile salt, cholesterols, carotenoids, and many others.

In a specific exemplary embodiment, the composition can comprise CoQ10, an enzyme that may CoQ10 treat and/or prevent migraine headaches, statin myopathy, kidney disease, infertility, skin aging, fatigue, diabetes, cancer, neurodegenerative diseases, and/or pulmonary conditions. The composition can comprise, for example, 50 to 500 mg/dosage of the composition, or about 100 to 200 mg/dosage.

In a specific exemplary embodiment, the composition can comprise cannabidiol (CBD). CBD may improve anxiety, cognition, insomnia, cardiovascular health and movement disorders, as well as reduce pain, IBS symptoms, and seizure activity. The composition can comprise, for example, from 10 to 1,500 mg/dosage of the composition, or about 20 to 100 mg/dosage.

In one embodiment, the composition is formulated as a dehydrated powder or concentrate that can be reconstituted into a drinkable fluid by the addition of water. In one embodiment, the composition is formulated as a blended smoothie or milkshake.

In one embodiment, the composition can be formulated for administering directly into the GI tract. For example, the composition can be formulated for administration to the proximal lower GI via colonoscopy, the distal lower GI via enema or rectal tubes, and the upper GI tract via nasogastric tubes, duodenal tubes, and endoscopy/gastroscopy.

The composition can be placed in containers of appropriate size, taking into consideration, for example, the intended use, the contemplated method of application, the size of the fermentation vessel, and any mode of transportation from microbe growth facility to the location of use. Thus, the containers into which the composition is placed may be, for example, from 0.1 gallon to 1,000 gallons or more. The composition may further be placed into containers, such as bottles, for distribution of individual doses of the composition.

### Methods

In preferred embodiments, the present invention further provides methods of enhancing the health of a subject in need thereof by, for example, restoring the subject's gut microbiome, increasing water and electrolyte absorption in the digestive tract, and/or providing a therapeutic substance to the subject to treat and/or prevent a disease, disorder or health condition.

In certain embodiments, the subject is dehydrated, or at risk of dehydration, due to, for example, physical exertion, heat exhaustion, a condition that causes vomiting or diarrhea, and/or as a side-effect of a medical treatment, such as a drug or radiation therapy.

In certain embodiments, the subject's gut microbiome is disrupted or imbalanced, or at risk of disruption or imbalance, due to, for example, infection by a pathogenic microorganism, illness, aging, dietary factors (e.g., food sensitivities, changes in eating and/or nutritional habits), immune system changes, treatment with antibiotics, radiation treatment or exposure, or procedures, such as appendectomies and/or colonoscopies.

In certain embodiments, the subject has, or is at risk of having, a disease, disorder or health condition, e.g., the cardiovascular system or the endocrine system, for example, diabetes (1 or 2) and/or cardiovascular disease.

In preferred embodiments, the present methods are used for enhancing and/or restoring the gut microbiome of a subject whose gut microbiome is in dysbiosis. The disruption or imbalance can be a result of, for example, infection by a pathogenic microorganism (e.g., H. pylori), illness, aging, dietary factors (e.g., food sensitivities, changes in eating and/or nutritional habits), immune system changes, radiation treatment or exposure, treatment with antibiotics, or procedures, such as appendectomies and/or colonoscopies.

As used herein, reference to the “microbiome,” “microbiota,” “microbial community,” “microflora” or “flora” of the “gut” or of the “GI tract” means the population of microorganisms living within a subject's intestines and/or GI tract. In some embodiments, these microorganisms can also be present in the appendix. A healthy, or balanced, gut microbiome is one that comprises a variety of microbial species, with a majority of those species preferably being beneficial to the health of the subject.

As used herein, a “beneficial” microbe is one that is considered mutualistic, or conferring a benefit to its host, rather than one that is merely commensal (existing within the gut in a non-harmful and non-mutualistic coexistence) or one that is harmful and/or parasitic to the host. Benefits can include, for example, digestion of dietary fiber into short-chain fatty acids and synthesis of certain vitamins.

As used herein, a “disrupted” or “unbalanced” gut microbiome is in dysbiosis, where the species of microbes in a subject's gut comprise an amount, percentage or number of non-beneficial microorganisms that results in disease, discomfort, malnutrition, impaired nutrient absorption and/or other deleterious health consequences. Non-beneficial microorganisms include harmful microorganisms, such as pathogens and parasites, as well as commensal organisms that do not directly harm the host, but when overgrown, outcompete beneficial, gut microorganisms.

As used herein, “restoring” a disrupted or unbalanced gut microbiome refers to establishing or reestablishing the predominance of beneficial microorganisms within the subject's gut microbial community, or causing the gut microbiome to become healthy and/or balanced. Restoring the gut microbiome can comprise balancing an unbalanced gut microbiome, regardless of whether the imbalance is a cause or an effect of a disease or another change to the subject's health status. Restoration preferably comprises decreasing the number of overgrown commensal microorganisms, pathogenic microorganisms and/or biofilms in the GI tract, and/or increasing the number of beneficial microorganisms in the GI tract.

In certain embodiments, the beneficial microorganisms and/or microbial growth by-products of the ingestible composition, outcompete and/or directly control undesirable gut microorganisms, thus restoring the gut microbiome to a healthy, balanced state.

In certain embodiments, the methods comprise administering a composition of the present invention to the subject, preferably by ingestion. The optimal rate of administration can depend upon, for example, the physiological characteristics of the subject, the nature or extent of dehydration and/or gut microbiome dysbiosis, and the existence and/or extensiveness of other health conditions. An exemplary rate of application would be approximately an 8 to 24 fluid ounce dosage every 4 hours to every 24 hours.

In some embodiments, the methods result in improved hydration and/or nutrient absorption for a subject who is dehydrated and/or depleted of a certain nutrient, or is at risk of becoming dehydrated and/or nutrient-depleted, due to, for example, prolonged physical activity, illness, or cancer treatments. Advantageously, in one embodiment, the amphiphilic molecules of the rehydration composition help bind to water molecules and electrolyte ions and transport them across the intestinal epithelial cells. Thus, in some embodiments, the methods help a subject achieve enhanced hydration by increasing water and electrolyte absorption in the subject's digestive tract.

In certain embodiments, the present invention can be used to enhance a subject's overall health and well-being. In one embodiment, the present invention can be used to enhance the functioning of a body system, tissue or organ, such as metabolic functions, the digestive system, the immune system, the endocrine system, and/or the cardiovascular system.

In a specific embodiment, the methods result in improved cardiovascular health due to, for example, the inclusion of nattokinase in the ingestible formulation. Advantageously, nattokinase is thought to benefit cardiovascular health through reduction in artherosclerotic plaques, reduction in blood pressure, fibrinolysis and/or improved blood circulation.

In some embodiments, the present invention can be used to treat and/or ameliorate the symptoms of health conditions that are a cause and/or a result of a disrupted or unbalanced gut microbiome, such as, for example, dehydration, Irritable Bowel Syndrome, Type 1 diabetes, Celiac disease, other autoimmune disorders, colorectal cancer, and neurodevelopmental and neurodegenerative diseases, such as ALS, ASD and Alzheimer's disease.

In one embodiment, the present invention can be used to treat and/or ameliorate digestive conditions and/or symptoms such as, for example, dehydration, nausea, vomiting, diarrhea, constipation, gas, bloating, food sensitivities, heartburn, acid-reflux, GERD, indigestion, and abdominal cramps/pain. In one embodiment, the present invention can be used to treat and/or ameliorate extra-intestinal symptoms associated with a variety of health conditions, including symptoms such as, for example, headaches, dizziness, fatigue, backaches, insomnia, eating disorders, nutrient deficiencies, depression, anxiety, fertility issues, joint or muscle pain, brain fog, genital yeast infections, bacterial vaginosis, bladder or urinary tract infections, and many others.

Intestinal bacteria are the most important components of mammalian metabolic and digestive processes. These microbes aide in enterohepatic circulation, and help with the digestion and assimilation of energy sources and essential trace elements into the GI system. Additionally, anaerobic bacteria of the gut break down and/or ferment heavy carbohydrate compounds, such as dietary fiber, into short-chain fatty acids, such as acetate, propionate and butyrate. Gut microbes also aide in the regulation of fat storage, blood sugar levels, and metabolism of several vitamins. For example, the intestinal microflora synthesizes vitamin K, which is a necessary cofactor in the production of prothrombin and other coagulation factors. Additionally, intestinal bacteria synthesize biotin, vitamin B12, folic acid and thiamine.

There is also a relationship between the gut microbiota and the immune system. The human intestine contains more immune cells than all other parts of the body. A balanced gut microbiome regulates many functions of the immune system, including the activation and regulation of immune cells, proliferation of regulatory T-cells, neutrophil activation, and migration of the monocytes and macrophages. Thus, disturbances in the gastrointestinal tract may lead to immune disorders, infections and mis-regulation of pro- and anti-inflammatory processes. Furthermore, various other health concerns, ranging from autoimmune diseases to clinical depression and obesity, can be related to immune dysfunction, which can be linked to an unbalanced gut microbiome.

A balanced gut microbiome is also important for hormone regulation. For example, serotonin, which is a hormone that participates in processes such as sleep, mood, sexual affection, production of breast milk, respiration, communication, production and regulation of melatonin and adrenaline, and many others, is largely (i.e., more than 90%) produced in the GI tract by enterochromaffin cells. The microbial makeup within the GI tract may play a role in proper serotonin production and regulation.

Additionally, an imbalance of the GI microbiome can affect the cardiovascular system. Disruptions in the gut microbiome contribute to an increase in the appearance of plaques and the progression of atherosclerosis. Furthermore, the GI tract affects the regulation of insulin, the resistance to which can lead to diabetes—a condition that greatly increases the risk of cardiovascular disease. Even further, disrupted gut flora can lead to accumulation of adipose tissue in organs such as the liver and heart, which also increases the risk of diseases associated with these organs.

In addition to the digestive, metabolic, endocrine and cardiovascular systems, changes in the gut microbiome are also associated with a number of autoimmune diseases, and may be a cause and/or an effect of these pathologies. For example, altered microbiota are thought to play a role in initiation and progression of inflammatory bowel syndrome (IBS), Crohn's disease, and ulcerative colitis through triggering of autoimmune responses and inflammation. The autoimmune disease, celiac disease, may also be triggered by bacterial and/or viral infections that augment gut mucosal responses to gluten. Furthermore, Type 1 diabetes is also caused by immune system alterations, which may be caused and/or progressed by gut microbiome alterations that affect the mucosal lining of the intestines and gut permeability.

The intestinal microbiome may also be key to the etiology of colorectal cancer in at least two ways: through the pro-carcinogenic activity of microbial pathogens and through the influence of the metabolome. For instance, suppression of inflammation and cancerous cells may be achieved by the presence of short-chain fatty acids, acetate, propionate and butyrate produced by beneficial gut microbes, while other compounds, such as secondary bile acids, may induce carcinogenesis. Pathogenic microbes may also cause DNA alterations that could lead to oncologic diseases.

There is also evidence for an association between altered gut microbiota and neurodevelopmental (e.g., autism) and neurodegenerative (e.g., Alzheimer's) diseases. For example, Alzheimer's disease is characterized by accumulation of amyloid-β fibrils in the brain. Gut microbiota were shown to produce a significant amount of amyloids and lipopolysaccharides, which affect the neural pathways and production of cytokines, thus contributing to the disease.

Additionally, anxiety and sensory sensitivity experienced by patients with autism have been found to correlate with GI problems, and in fact, about 70% of children with autism have GI problems. Furthermore, children with autism show a higher level of Clostridium histolyticum than healthy children, and certain microbes, such as Desulfovibrio bacteria may play a role in the development of regressive autism.

In some embodiments, the present methods comprise analyzing the subject's health before and after administration of the composition to determine the effectiveness of the composition.

A subject's hydration levels can be analyzed using, for example, electrolyte blood tests, urinalysis, body weight measurements, urination frequency measurements, and other methods known in the medical arts.

In one embodiment, a subject's gut microbiome can be analyzed by taking a sample from the subject's GI tract, wherein the sample comprises a microbial community. In one embodiment, the sample is taken from the subject's appendix. In one embodiment, the sample comprises a representation of the entire microbial community within the subject's GI tract.

The sample can be collected by means known in the medical arts. For example, the sample can be a stool sample, intestinal mucosal lavage samples, and/or an intestinal and/or appendix tissue specimen collected via endoscopy and/or biopsy.

The sample is then analyzed to identify microbial species present within the GI microbial community, and to determine the population percentage of each species with respect to each of the other species of the microbial community. Analysis can comprise standard methods in the art, such as, for example, DNA sequencing, DNA fingerprinting, ELISA, and cell plating.

### Growth of Microbes According to the Present Invention

In one embodiment, the present invention provides methods for cultivation of microorganisms and production of microbial metabolites and/or other by-products of microbial growth using a novel form of solid state, or surface, fermentation. Hybrid systems can also be used. As used herein “fermentation” refers to growth of cells under controlled conditions. The growth could be aerobic or anaerobic.

In one embodiment, the present invention provides materials and methods for the production of biomass (e.g., viable cellular material), extracellular metabolites (e.g. small molecules, polymers and excreted proteins), residual nutrients and/or intracellular components (e.g. enzymes and other proteins).

The microbe growth vessel used according to the present invention can be any enclosed fermenter or cultivation reactor for industrial use. In one embodiment, the reactor is a proofing oven, such as a standard oven used in commercial baking for, e.g., proofing dough. In one embodiment, the reactor is in the form of a scaled-up enclosure, such as a trailer or a room, that is equipped with the necessary components to provide, for example, tens or hundreds of trays of culture growing on matrix to be incubated at the same time. In one embodiment, the reactor can optionally be equipped with an automated conveyor system or pulley system for continuous production.

In one embodiment, the vessel may optionally have functional controls/sensors or may be connected to functional controls/sensors to measure important factors in the cultivation process, such as pH, oxygen, pressure, temperature, agitator shaft power, humidity, viscosity and/or microbial density and/or metabolite concentration. Preferably, no such controls are necessary, however.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of microbes in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. The oxygenated air may be ambient air supplemented daily through, e.g., air pumps.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors, trace nutrients and/or biostimulants for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate (e.g., ferrous sulfate heptahydrate), iron chloride, manganese sulfate, manganese sulfate monohydrate, manganese chloride, zinc sulfate, lead chloride, copper sulfate, calcium chloride, calcium carbonate, and/or sodium carbonate. These inorganic salts may be used independently or in a combination of two or more.

In some embodiments, the method for cultivation may optionally comprise adding additional acids and/or antimicrobials in to the substrate before and/or during the cultivation process. Advantageously, however, the subject method reduces or eliminates the need for protection from contamination during cultivation due in part to the slower rate of microbial growth.

The pH of the mixture should be suitable for the microorganism of interest, though advantageously, stabilization of pH using buffers or pH regulators is not necessary when using the subject cultivation methods.

The method and equipment for cultivation of microorganisms and production of the microbial by-products can be performed in a batch process or a quasi-continuous process.

In one embodiment, the method for cultivation of microorganisms is carried out at about 15 to 60° C., preferably, 25 to 40° C., and in specific embodiments, 25 to 35° C., or 32 to 37° C. In one embodiment, the cultivation may be carried out continuously at a constant temperature. In another embodiment, the cultivation may be subject to changing temperatures. Temperature can be kept within the preferred range by pumping ambient air into the reactor and circulating it throughout.

In one embodiment, total sterilization of equipment and substrate used in the subject cultivation methods is not necessary. However, the equipment and substrate can optionally be sterilized. The trays can be sterilized before and/or after being spread with nutrient medium, for example, using an autoclave. Additionally, the steam pan lids and pan bands can be sterilized, for example, by autoclaving, prior to inoculation of the solid substrate.

The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control bacterial growth.

In one embodiment, the present invention further provides methods of producing a microbial metabolite by cultivating a microbe strain under conditions appropriate for growth and metabolite production. Optionally, the method can comprise purifying the metabolite. The present invention provides methods of producing metabolites such as, e.g., biosurfactants, biopolymers, ethanol, lactic acid, beta-glucan, proteins, peptides, metabolic intermediates, polyunsaturated fatty acid, lipids and enzymes.

The microbial growth by-product produced by microorganisms of interest may be retained in the microorganisms or secreted into the substrate. The metabolite content can be, for example, at least 20%, 30%, 40%, 50%, 60%, 70%, 80%, or 90%.

In another embodiment, the method for producing microbial growth by-product may further comprise steps of concentrating and purifying the microbial growth by-product of interest. In a further embodiment, the substrate may contain compounds that stabilize the activity of microbial growth by-product.

In one embodiment, all of the microbial cultivation composition is removed upon the completion of the cultivation (e.g., upon, for example, achieving a desired spore density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a cell-free substrate or contain cells. In this manner, a quasi-continuous system is created.

In one embodiment, the systems and methods of the present invention can be used to produce a microbial metabolite, wherein instead of drying the microbial slurry, the microbial slurry is filtered to separate the liquids from the solids. The liquid that is extracted, which comprises the microbial metabolite, can then be purified further, if desired, using, for example, centrifugation, rotary evaporation, microfiltration, ultrafiltration and/or chromatography.

The metabolite and/or growth by-product can be, for example, a biosurfactant, enzyme, biopolymer, acid, solvent, amino acid, nucleic acid, peptide, protein, lipid and/or carbohydrate. Specifically, in one embodiment, the method can be used to produce a biosurfactant.

Advantageously, the method does not require complicated equipment or high energy consumption. The microorganisms of interest can be cultivated at small or large scale on site and utilized, even being still-mixed with their media. Similarly, the microbial metabolites can also be produced at large quantities at the site of need.

Advantageously, the microbe-based products can be produced in remote locations. The microbe growth facilities may operate off the grid by utilizing, for example, solar, wind and/or hydroelectric power.

### Local Production of Microbe-Based Products

In certain embodiments of the present invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the present invention can be located at the location where the microbe-based product will be used. For example, the microbe growth facility may be less than 300, 250, 200, 150, 100, 75, 50, 25, 15, 10, 5, 3, or 1 mile from the location of use.

The microbe growth facilities of the present invention produce fresh microbe-based compositions comprising the microbes themselves, microbial metabolites, and/or other components of the medium in which the microbes are grown. If desired, the compositions can have a high density of vegetative cells or propagules, or a mixture of vegetative cells and propagules.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. The system is efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the present invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with a subject's health conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific subject and the subject's health conditions present at the time of application. This provides advantages over compositions that are pre-made in a central location and have, for example, set ratios and formulations that may not be optimal for a given subject.

The microbe growth facilities provide manufacturing versatility by their ability to tailor the microbe-based products to improve synergies with unique subjects. Advantageously, in preferred embodiments, the systems of the present invention harness the power of naturally-occurring gut microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### REFERENCES

Clemente, J. C., Ursell, L. K., Parfrey, L. W., & Knight, R. (2012). The Impact of the Gut Microbiota on Human Health: An Integrative View. Cell, 148(6), 1258-1270. doi:10.1016/j.cell.2012.01.035. (“Clemente 2012”).

Turnbaugh, P. J., Quince, C., Faith, J. J., McHardy, A. C., Yatsunenko, T., Niazi, F., Affourtit, J., Egholm, M., Henrissat, B., Knight, R., and Gordon, J. I. (2010). Organismal, genetic, and transcriptional variation in the deeply sequenced gut microbiomes of identical twins. Proc. Natl. Acad. Sci. USA 107, 7503-7508. (“Turnbaugh 2010”).

Tannock, G. W. (2003). The Intestinal Microflora. In R. Fuller & G. Perdigon (Eds.), Gut Flora, Nutrition, Immunity and Health (pp. 1-23). Malden, USA: Blackwell Publishing Ltd. (“Tannock 2003”).

Lozupone, C., Stombaugh, J., Gordon, J., Jansson, J., & Knight, R. (2012). Diversity, stability and resilience of the human gut microbiota. Nature, 489(7415), 220-230. doi: 10.1038/nature11550. (“Lozupone 2012”).

Benson, A. K., Kelly, S. A., Legge, R., Ma, F., Low, S. J., Kim, J., Zhang, M., Oh, P. L., Nehrenberg, D., Hua, K., et al. (2010). Individuality in gut microbiota composition is a complex polygenic trait shaped by multiple environmental and host genetic factors. Proc. Natl. Acad. Sci. USA 107, 18933-18938. (“Benson 2010”).

Nagpal R, Mainali R, Ahmadi S, et al. Gut microbiome and aging: Physiological and mechanistic insights. Nutrition and Healthy Aging. 2018; 4(4):267-285. doi:10.3233/NHA-170030. (“Nagpal 2018”).

1. A method for enhancing a subject's health, the method comprising administering to the subject a composition comprising water, a biosurfactant,
electrolytes,
a beneficial microorganism, and an additive
selected from nutrients, vitamins, minerals, herbal extracts, proteins, amino acids, antioxidants, and health supplements,
wherein the method results in an increase in water and/or electrolyte absorption in the subject's digestive tract, restoration of the subject's gut microbiome, and/or providing a therapeutic substance to the subject.

2. (canceled)

3. The method of claim 1, comprising a biosurfactant selected from glycolipids, lipopeptides and/or phospholipids.

4. The method of claim 1, wherein the composition is administered to the subject via ingestion.

5. The method of claim 1, comprising electrolytes selected from sodium, potassium, phosphate, bicarbonate, sulfate, chloride, calcium and/or magnesium.

6. The method of claim 1, comprising a beneficial microorganism selected from Bacillus spp., Bacteroides spp., Bifidobacterium spp., and Lactobacillus spp.

7. The method of claim 6, wherein the microorganism is selected from Bacillus subtilis natto, Bacillus amyloliquefaciens or Bacillus coagulans.

8. The method of claim 1, comprising one or more additives selected from nattokinase, CoQ10 and cannabidiol.

9. (canceled)

10. The method of claim 1, wherein the subject's gut microbiome has been disrupted or unbalanced as a result of antibiotic treatment, illness, infection, dietary factors, radiation treatment or exposure, colonoscopy, appendectomy, and/or aging.

11. The method of claim 1, wherein the subject is dehydrated due to physical exertion, heat exhaustion, drug side-effects, or a condition that causes vomiting or diarrhea.

12. The method of claim 1, wherein the functioning of the digestive system, immune system, endocrine system or cardiovascular system is enhanced.

13. The method of claim 12, wherein the cardiovascular system is enhanced via dissolving of artherosclerotic plaques, improving of blood circulation, lowering of blood pressure, and/or fibrinolysis.

14. The method of claim 1, symptoms of Irritable Bowel Syndrome, Type 1 diabetes, Celiac disease, colorectal cancer, neurodevelopmental diseases or neurodegenerative diseases are ameliorated.

15. The method of claim 1, wherein digestive conditions and/or digestive symptoms selected from dehydration, nausea, vomiting, diarrhea, constipation, gas, bloating, food sensitivities, heartburn, acid-reflux, GERD, indigestion, and abdominal cramps/pain are ameliorated.

16. The method of claim 1, wherein extra-intestinal symptoms associated with a health condition, wherein the symptoms are selected from headaches, dizziness, fatigue, backaches, insomnia, eating disorders, nutrient deficiencies, depression, anxiety, fertility issues, joint or muscle pain, brain fog, genital yeast infections, bacterial vaginosis, and bladder or urinary tract infections are ameliorated.

17. An ingestible composition comprising water, a biosurfactant,
an electrolyte,
a beneficial microorganism, and
one or more additives selected from nutrients, vitamins, minerals, herbal extracts, proteins, amino acids, antioxidants, and health supplements.

18. (canceled)

19. The composition of claim 17, comprising a biosurfactant selected from glycolipids, lipopeptides and/or phospholipids.

20-21. (canceled)

22. The composition of claim 17, wherein the one or more beneficial microorganisms are Bacillus spp., Bacteroides spp., Bifidobacterium spp., and/or Lactobacillus spp.

23. The composition of claim 22, wherein the microorganism is selected from Bacillus subtilis natto, Bacillus amyloliquefaciens or Bacillus coagulans.

24. The composition of claim 17, comprising one or more additives selected from nattokinase, CoQ10 and cannabidiol.
